(19) United States
(12) Patent Application Publication
(10) Pub. No.: US 2010/0221723 A1
(43) Pub. Date: Sep. 2, 2010

(54) EARLY DETECTION OF CANCER BY METHYLATED DNA IN BLOOD

(75) Inventors: Regina Santella, Teaneck, NJ (US); Yu-Jing Zhang, New York, NY (US); Chien Jen Chen, Taipei (TW); Jing Shen, Tenafly, NJ (US)

Correspondence Address: LAW OFFICES OF ALBERT WAI-KIT CHAN, PLLC, 141-07 20th AVENUE, WORLD PLAZA, SUITE 604, WHITESTONE, NY, 11357, US

(73) Assignee: THE TRUSTEES OF COLUMBIA UNIVERSITY IN THE CITY OF NEW YORK, New York, NY (US)

(21) Appl. No.: 12/673,416

(22) PCT Filed: Aug. 13, 2008
(86) PCT No.: PCT/US08/73039

§ 371 Date: May 14, 2010

Related U.S. Application Data
(60) Provisional application No. 60/955,733, filed on Aug. 14, 2007.

(51) Int. Cl. C12Q 1/68

(52) U.S. Cl. 435/6

(57) ABSTRACT

This invention relates to early detection of cancer by detecting methylated DNA in blood. In one embodiment, there is provided a method of predicting the occurrence of hepatocellular carcinoma in a subject, comprising the steps of preparing DNA samples from blood samples of the subject; and determining methylation status of a group of genes comprising RASSF1A, p16 and p15, wherein hypermethylation of these genes as compared to normal control samples indicates the subject is likely to develop hepatocellular carcinoma in the future.

### CROSS-REFERENCE TO RELATED APPLICATIONS

This application claims the benefit of priority of U.S. Application Ser. No. 60/955,733, filed Aug. 14, 2007. The entire contents and disclosure of the preceding application is incorporated by reference into this application.

Throughout this application, various references or publications are cited. Disclosures of these references or publications in their entireties are hereby incorporated by reference into this application in order to more fully describe the state of the art to which this invention pertains.

### FIELD OF THE INVENTION

This invention relates to early detection of cancer by methylated DNA in blood.

### BACKGROUND OF THE INVENTION

Hepatocellular carcinoma (HCC) is one of the most common and rapidly fatal human malignancies. The almost 500,000 new cases and nearly equivalent number of fatalities illustrates the lack of effective therapeutic alternatives for this disease that is largely diagnosed at an advanced stage; most patients die within one year of diagnosis. Chronic hepatitis B and C virus infections are well-documented risk factors for the development of HCC. Several environmental factors including aflatoxin B₁ (AFB₁), a dietary mold contaminant, and polycyclic aromatic hydrocarbons, ubiquitous environmental contaminants, are also associated with the development of HCC. While HCC incidence is highest in East Asia and Sub-Saharan Africa, it is also increasing in U.S. Currently available screening tests to detect smaller and more frequently unifocal (early stage) HCC combine alpha-fetoprotein (AFP) analysis and ultrasound. However, although screening for early detection of HCC has become quite common in clinical practice, its effectiveness remains controversial.

As with other cancers, the development of HCC is a complex, multistep process. The molecular pathogenesis of HCC appears to involve multiple genetic aberrations in the molecular control of hepatocyte proliferation, differentiation, and death and the maintenance of genomic integrity. This process is influenced by the cumulative activation and inactivation of oncogenes, tumor suppressor genes and other genes. Epigenetic alterations are also involved in cancer development and progression. Methylation of promoter CpG islands is known to inhibit transcriptional initiation and cause permanent silencing of downstream genes. Hypermethylation of p16, a cyclin-dependent kinase inhibitor gene that regulates the cell cycle, has been detected frequently in human cancers. p15, another cyclin-dependent kinase inhibitor gene adjacent to p16 on chromosome 9p21, has been postulated to be a tumor suppressor modulating pRb phosphorylation. It is also aberrantly methylated in several human neoplasms including HCC. The ras association domain family 1A (RASSF1A) gene is located in chromosome 3p21.3 and, from initial studies in lung and breast cancer, was suggested to be a tumor suppressor gene. RASSF1A promoter hypermethylation in HCC tissues has been consistently reported at a high frequency, in the range of 80-90%.

Detection of methylated DNA has been suggested as a potential biomarker for early detection of cancer. Since an ideal biomarker should appear early in the course of disease and should be detectable in biological samples that can be obtained noninvasively, many studies have focused on the detection of genetic and epigenetic abnormalities in exfoliated cells from sputum, bronchoalveolar lavage or cervical smears as well as in the circulating DNA found in serum or plasma. In a recent study, p16 was methylated in 24 of 40 (62%) tissues and 12 of 39 (32%) plasma DNAs from blood collected at the time of diagnosis. Other studies of HCC patients have also found that methylated DNA can be frequently detected in blood collected at the time of diagnosis. These results suggest that biomarkers in plasma or serum may help in estimating the risk for the development of HCC, however, their sensitivity and specificity for HCC detection, and their clinical utility remain uncertain at the present time. No studies of HCC have assayed blood samples collected years prior to diagnosis for aberrant methylation.

### SUMMARY OF THE INVENTION

Aberrant gene expression is the hallmark of cancer cells. In addition to classical genetic mechanisms such as deletions and mutations, growth regulatory genes can be inactivated epigenetically via methylation of cytosine-residues in the promoter region of these genes. Hypermethylation of CpG islands in promoter regions is now recognized as an important and early event in carcinogenesis. Detection of methylated DNA in serum or plasma has been suggested to be a marker for early cancer development.

Data presented below are from the first study to prospectively examine epigenetic changes in tumor suppressor genes for predicting hepatocellular carcinoma (HCC) development in a cohort of high-risk subjects. p16, p15 and RASSF1A promoter hypermethylation were detected in DNA from serum samples collected up to 9 years before clinical diagnosis. Compared to controls, detection of promoter hypermethylation on these three genes was much more frequent in HCC patients prior to diagnosis. These molecular changes may be a valuable biomarker for early detection, risk assessment in high-risk populations and monitoring the clinical course of HCC.

The present invention also determines if plasma DNA can be used for the early diagnosis of prostate cancer. The present invention also examines whether tumor DNA can be detected in plasma of blood collected prior to diagnosis of breast cancer in women and their healthy siblings from high risk families.

### BRIEF DESCRIPTION OF THE DRAWINGS

FIG. 1 shows RASSF1A and p16 methylation status of serum DNA at different time points prior to diagnosis of HCC. MSP data using methylation-specific primers for RASSF1A and p16 are shown for five cases. PCR products were stained with Vista Green after agarose gel electrophoresis. The size of the PCR products is 93 bp for RASSF1A and 145 for p16.

FIG. 2 shows p15 methylation status of serum DNA at different time points prior to diagnosis of HCC. MSP data using methylated (m)- and unmethylated (u)-specific primers for p15 are shown for four cases. PCR products were stained with Vista Green during agarose gel electrophoresis. The sizes of the PCR products for methylated and unmethylated primers are 154 and 162 bp, respectively.

FIG. 3 shows receiver-operator characteristic (ROC) curves of sensitivity versus 1—specificity. A: ROC curve for the model that includes the predictive variables, age, HBsAg status, anti-HCV status, smoking status, and alcohol consumption. The overall predictive accuracy is 67% for the probability cut-point of 0.50. Sensitivity=66%; Specificity=68%. B: ROC curve for the model that includes all the variables in A plus the three hypermethylation biomarkers (p16, p15 and RASSF1). The overall predictive accuracy is 89% for the probability cut-point of 0.50. Sensitivity=84%; Specificity=94%

### DETAILED DESCRIPTION OF THE INVENTION

The present invention provides a method of predicting the occurrence of hepatocellular carcinoma in a subject, comprising the steps of: preparing DNA samples from blood samples of the subject; and determining methylation status of a group of genes comprising RASSF1A, p16 and p15, wherein hypermethylation of these genes as compared to normal control samples indicates the subject is likely to develop hepatocellular carcinoma in the future. In general, the blood samples are serum or plasma samples. The method may be performed at least one year before the occurrence of hepatocellular carcinoma in the subject, and the subject may be in a high risk group for developing hepatocellular carcinoma. In one embodiment, the hypermethylation occurs at the promoter regions of the genes.

The present invention also provides a method of predicting the occurrence of breast cancer in a subject, comprising the steps of: preparing DNA samples from blood samples of the subject; and determining methylation status of a group of genes comprising RASSF1A, and p16, wherein hypermethylation of these genes as compared to normal control samples indicates the subject is likely to develop breast cancer in the future. In general, the blood samples are serum or plasma samples. The method may be performed at least one year before the occurrence of breast cancer in the subject. In one embodiment, the hypermethylation occurs at the promoter regions of the genes.

The present invention also provides a method of predicting the occurrence of prostate cancer in a subject, comprising the steps of: preparing DNA samples from blood samples of the subject; and determining methylation status of one or more genes from a group comprising 1) GSTP1, 2) RASSF1A, 3) RARβ2, 4) APC, 5) p16, 6) TNFRSF10C, 7) BCL2, 8) MDR1, 9) ASC, 10) MGMT, 11) DAPK, 12) MT1G, 13) CDH1, 14) PTGS2 and 15) TIG1, wherein hypermethylation of these genes as compared to normal control samples indicates the subject is likely to develop prostate cancer in the future. These genes may be used alone or in any combination thereof; a combination of genes may thus comprise genes from any or all of the genes listed above as 1-15 in any permutation, e.g. may include genes 1-5, 1-10, 1-15, or genes 3, 4, and 5. In general, the blood samples are serum or plasma samples. The method may be performed at least one year before the occurrence of prostate cancer in the subject. In one embodiment, the hypermethylation occurs at the promoter regions of the genes.

### Example 1

### Genetic and Epigenetic Alterations in Tumors

Over the past few years, data on the detection of tumor DNA in blood has increased rapidly¹⁻⁶. These studies provide hope that new sensitive methods for early detection of cancer may be feasible. There is still much work to be done before these methods become clinically useful, including determination of the sensitivity and specificity of detection and the time frame before clinical diagnosis in which the markers becomes detectable. The CSP sample bank provides us with an excellent opportunity to investigate tumor DNA in blood for two long term goals, to collect information on HCC etiology and also to help in its early diagnosis.

As detailed below, we are carrying out a pilot study comparing tumor and blood DNA alterations (mutations in p53 and methylation of the CpG promoter region in p16) in a set of samples collected from newly identified cases not part of the CSP cohort. This is being followed by the analysis of blood collected 1-2 years before diagnosis for 50 CSP HCC cases. p16 methylation was found in plasma DNA of 44% of samples (see data below). These bloods are available because high risk cohort members (see details in Research Design) are followed intensively as part of the cancer screening program with yearly blood tests for determination of liver function. Thus, we will obtain information about the time frame in which tumor alterations become detectable in blood. We are also exploring the relationship between environmental exposures and DNA alterations, recognizing that this small sample size will not provide power for definitive conclusions.

We now propose to carry out the next step, a nested case-control analysis of plasma DNA. While we initially analyzed both p53 mutations and methylation in p16, because of the ease of the MethyLight assay³ (described below), only analysis of gene-specific hypermethylation will be used in future studies. We also propose to increase the number of genes to be studied. Again this is made feasible by the MethyLight assay that is carried out in 96 well plates. The selection of genes to be analyzed is based on published data and our data on specific genes found to be methylated at reasonably high frequencies in HCC, as well as data on methylation in tumors at other sites. The goal of these studies is to select a panel of genes that will be methylated more frequently in cases compared to controls. A second goal is to explore the relationship between environmental exposures and methylation.

Because of space limitations, we provide only a brief description of the genes and do not describe in detail their biological functions. More detail for some genes can be found in our published papers in the Appendix. Selection was based on high frequency of methylation in HCC tissues or plasma and low frequency in control plasma, but with the caveat that data on controls are not currently available for several genes. The p16^{INK4} gene, encoding a G1-specific cell cycle inhibitor, is methylated in 30-70% of HCCs^{7,8}. Our own studies, described below, found 47 and 62% methylation in two sets of HCC samples from Taiwan⁹ and unpublished data in Progress Report. We and others have also determined the frequency of tumor DNA in blood containing methylated p16. Approximately 80% of subjects with methylation positive tumors also had methylated DNA in blood^{5,6}. Our data indicated ˜50% positive. In our pilot study, we found 44% of plasma DNAs positive 1-2 years prior to diagnosis. Others found no changes in 22 bloods from HCC patients without tumor alterations or in 38 patients with chronic hepatitis/cirrhosis or in 10 healthy controls⁵. We found one plasma sample positive when the tumor was negative.

Ras association domain family 1 (RASSF1), cloned from the lung tumor suppressor locus 3p21.3, is methylated in a number of tumors¹⁸⁴⁻¹⁸⁷, including HCC^{9,14}. Both our study¹⁴ and the other⁹ found methylation in approximately 85% of HCCs. There are no data on blood DNA in HCC. However, a breast cancer study that also analyzed blood from 10 healthy controls found methylation in 10% of controls compared to 39% of 26 primary cases and 80% of 10 recurrent cases¹. So although this gene is highly methylated in tumors, it may not be a specific marker. Studies in HCC with relatively large numbers of cases and controls are needed.

GSTP1 methylation has been found in approximately 50-90% of HCC tumors by us and others¹⁵⁻¹⁷. O⁶-methylguanine-DNA methyltransferase (MGMT) is a repair protein that specifically removes promutagenic alkyl groups from the O⁶ position of guanine in DNA. We found ˜40% of HCC tumors methylated. β-catenin interacts with E-cadherin at the plasma membrane as part of the Wnt signal transduction pathway. Its turnover is mediated by phosphorylation and ubiquitin-mediated degradation via the APC-Axin-GSK3b complex. E-cadherin and APC are methylated in about 50% of HCC¹⁷. SOCS-1 (65%) and p15 (49%) were also found to be among the most frequently methylated genes in HCC¹⁷⁻¹⁹. SOCS-1 is a protein that suppresses the JAK/STAT pathway by rendering cells unresponsive to cytokine stimulation. P15 encodes a cyclin-dependent kinase inhibitor and methylated DNA has been observed in 25% of HCC patient's plasma¹⁸. For the other genes, to the best of our knowledge, there are no data on methylation in blood DNA in HCC. But a study of gastric cancer detected methylation of E-cadherin, GSTP1, p15 and p16 in 57, 15, 56 and 52% of case bloods but none of the 30 controls²⁰.

### Cancer Screening Program (CSP) Cohort—Study Design

This cohort was originally set up for the evaluation of cancer screening efficacy. Study subjects were voluntary participants in a free cancer screening program implemented in seven urban and rural townships including San-Chi, Chu-Tung, Pai-Hsa, Hu-Hsi, Ma-Kung, Pu-Tze and Kao-Su. These townships represent a wide range of liver cancer mortality. Pai-Hsa, Hu-Hsi and Ma-Kung are located in Penghu Islets where the rate of HCC is extraordinarily high and our urine and albumin adduct data have documented high aflatoxin exposure. A total of 12,024 men and 13,594 women aged 30-64 were recruited in 1990-1992. Individuals were selected utilizing the household registration system that has been in operation since the 1930s. Every birth, death, marriage, divorce, education and employment has been registered. In addition to the cancer screening, the program included personal interviews using a structured questionnaire to obtain baseline information on sociodemographic characteristics, alcohol drinking (starting age, duration and quantity), cigarette smoking, dietary consumption (meals per week) of various food categories including pickled vegetables, cured meats, fermented foods, salted foods and animal liver, and family history of liver disease. For each subject, 15 ml of blood was collected using heparinized tubes, and buffy coat, plasma and red blood cells separated and stored at −70°. Spot urines were also collected and stored. As part of the screening program, all bloods were analyzed for HBV and HCV (HBsAg and anti-HCV kits from Abbott Laboratories). Bloods were also analyzed for α-fetoprotein, alanine transaminase and aspartate transaminase. High risk subjects (positive for at least one of the assays or with a family history of HCC or cirrhosis among first degree relatives, n=4,262) received ultrasound for early detection of HCC. As cases and controls were interviewed before the development of HCC, the potential for information bias is virtually none.

The second and third follow-up of the cohort was carried out between 1992 and 1995 by phone and mail contact. Subjects were asked to visit the local health station or Provincial Public Hospital where they were recruited at which time a structured questionnaire soliciting information on occupation, change in smoking and drinking habits and health status was administered. Blood and urine samples were again obtained. Approximately 60% of subject completed this follow up. Telephone interviews were carried out to obtain information on health status and hospitalization for those unable to attend. The approximately 4300 subjects at high risk have been actively followed from 1999-2004 again by phone and mail request to come in for a blood draw and ultrasound of the liver. Copies of death certificates in the study areas are obtained periodically from the local housing offices and intensive follow-up was accomplished through linkage with the national death certification and cancer registration data bases, using national identification number, sex and birth date. The overall follow-up rate was 98%. Through June 2004, 282 cases were identified in the CSP cohort. We had anticipated 350 by June 2005 but now believe 325 is a more accurate estimate with 450 by the end of year 4 of the current proposal (Jun. 30, 2009). We will continue to match cases to 5-6 controls to ensure biospecimens for two HBsAg positive and two HBsAg negative controls. Matching will be based on age (within 3 years), sex and time of blood collection (within 3 months).

Plasma, urine and white blood cells will be pulled from storage for shipment to Columbia. While we have in the past used a student or postdoc to transport the samples, current CDC regulations for samples possibly containing HBV require declaration as hazardous goods and the use of a commercial service. We have had no difficulties in sample transport with this service for recent shipments.

Assay DNA Isolated from Plasma of Cases and Controls in the CSP Cohort for the Frequency of Methylation of p15, p16, RASSF1A, MGMT, GSTP, APC, SOCS-1 and E-cadherin.

As part of the cancer screening program, high risk subjects have blood drawn on a yearly basis for liver function assays. Since most new HCC cases arise in these subjects, annual blood samples for a number of years prior to diagnosis are available from most cases. For aim 3, we will retrieve 200 plasma sample collected 1-2 years prior to diagnosis. The ideal control group would be healthy subjects in the cohort who provided blood samples in the same time frame. However, the CSP study only collected yearly blood samples from those at high risk for cancer development. Thus, two different types of controls will be used. One set of control samples will consist of 200 high risk subjects whose blood was collected within 6 months of that of the matched case. We will select subjects without cirrhosis. The second set of controls will be 200 currently healthy subjects not at high risk who provided baseline bloods. Both sets of controls will be matched to cases for gender, age at blood donation and HBV status. DNA will be isolated from 500 μl of plasma using Qiagen kits. Sodium bisulfite conversion will be carried out using the CpGnome DNA Modification Kit (Intergen, Purchase, N.Y.). Samples will be analyzed using the MethyLight assay as described³. Methylation specific primers for the genes of interest have already been reported^{1,3}. To normalize for input DNA, β-actin will be amplified. Specificity of the reactions for methylated DNA will be confirmed using human sperm DNA with low levels of methylation and SssI-treated DNA. The percentage of fully methylated molecules at a specific locus will be calculated as reported³ by dividing the gene:actin ratio of a sample by that of SssI-treated sperm DNA and multiplying by 100.

Limitations/alternate strategies The main limitation of this aim is that multiple bloods are not available from healthy, low risk subjects so that case and control bloods will have been stored for different periods of time. However, since DNA is a relatively stable molecule, we do not anticipate any problems with differing sample storage times on DNA yield. In the 50 cases in which we analyzed p16 methylation in plasma DNA, there was no relationship between time of sample collection and DNA yield. Multiple bloods are being collected from high risk subjects and will be used as an additional control group. These bloods will be stored for a similar time as the case bloods. Cirrhosis patients are already be partly down the pathway to HCC and may have increased methylation. It will be of interest to assay all bloods from those with cirrhosis in the future to determine if the panel of genes we select can predict cancer development. The single study to examine this found none of 38 patients had methylated p16 in plasma DNA⁵. Additional studies in larger numbers of subjects with cirrhosis and for a larger number of genes are warranted. We routinely monitor new publications on methylation in HCC and if new genes are identified that may be of use, we will incorporate them into the study. Finally, while we have not yet run the MethyLight assay on plasma DNA, this aim could be completed with methylation specific PCR as already done for p16.

The percentage of fully methylated DNA values obtained from the MethyLight assay will be dichotomized for statistical purposes. Previous publications used a cutoff of 4 since that gave the best discrimination between normal and malignant tissues across all CpG promoter region islands³. We will confirm that this is also appropriate for our study. Samples having ratios above 4 are designated methylated and given a value of 1 while those with lower ratios were given a value of 0. The statistical analyses of the matched case-control study will consider the risk of liver cancer (P(D)) in relation to frequency of methylation of p15, p16, RASSF1A, MGMT, GSTP, APC, SOCS-1 and E-cadherin. The intent of the analysis will be to build a model that will best predict the P(D). The following model will be considered

ln {P[D]/(1−P[D])}=α+β₁p15+β₂p16+β₃RASSF1A+β₄MGMT+β₅GSTP+β₆APC+β₇SOCS-1+β₈E-cadherin+ηsmoking+θalcohol+φ(other confounders)

Conditional likelihood will be used to obtain the estimates of the parameters. P₀ and P₁ are, respectively, the proportion of subjects with methylated DNA in controls and cases. Table 1 gives the study power for the main effect of the methylated DNA for varying P₀ and P₁ based on 200 cases and 200 controls. Power was calculated based on an unmatched case-control study. In general, our matched case-control study should have higher power.

TABLE 1
Power for Main Methylated DNA

| | | P₁ | | |
|---|---|---|---|---|
| Power | | .25 | .30 | .35 |
| P0 | .05 | 99% | 99% | 99% |
| | .10 | 97% | 99% | 99% |
| | .15 | 66% | 93% | 99% |

### REFERENCES

1. Muller, H. M., Widschwendter, A., Fiegl, H., Ivarsson, L., Goebel, G., Perkmann, E., Marth, C., and Widschwendter, M. DNA methylation in serum of breast cancer patients: an independent prognostic marker. Cancer Research, 63: 7641-7645, 2003.
2. Szymanska, K., Lesi, O. A., Kirk, G. D., Sam, O., Taniere, P., Scoazec, J. Y., Mendy, M., Friesen, M. D., Whittle, H., Montesano, R., and Hainaut, P. Ser-249TP53 mutation in tumour and plasma DNA of hepatocellular carcinoma patients from a high incidence area in the Gambia, West Africa. International Journal of Cancer, 110: 374-379, 2004.
3. Eads, C. A., Lord, R. V., Wickramasinghe, K., Long, T. I., Kurumboor, S. K., Bernstein, L., Peters, J. H., DeMeester, S. R., DeMeester, T. R., Skinner, K. A., and Laird, P. W. Epigenetic patterns in the progression of esophageal adenocarcinoma. Cancer Research, 61: 3410-3418, 2001.
4. Hogue, M. O., Begum, S., Topaloglu, O., Jeronimo, C., Mambo, E., Westra, W. H., Califano, J. A., and Sidransky, D. Quantitative detection of promoter hypermethylation of multiple genes in the tumor, urine, and serum DNA of patients with renal cancer. Cancer Research, 64: 5511-5517, 2004.
5. Gaudet, M. M., Britton, J. A., Kabat, G. C., Steck-Scott, S., Eng, S. M., Teitelbaum, S. L., Terry, M. B., Neugut, A. I., and Gammon, M. D. Intake of vegetables, fruits, and antioxidant vitamins and breast cancer among women in the Long Island Breast Cancer Study Project. Cancer Epidemiol. Biomarkers & Prev., 13: 1485-1494, 2004.
6. Wong, I. H., Zhang, J., Lai, P. B., Lau, W. Y., and Lo, Y. M. Quantitative analysis of tumor-derived methylated p16INK4a sequences in plasma, serum, and blood cells of hepatocellular carcinoma patients. Clinical Cancer Research, 9: 1047-1052, 2003.
7. Hui, A. M., Sakamoto, M., Kanai, Y., Ino, Y., Gotoh, M., and Yokota, J. Inactivation of p16^{INK4} in hepatocellular carcinoma. Hepatol., 24: 575-579, 1996.
8. Matsuda, Y., Ichida, T., Matsuzawa, J., Sugimura, K., and Asakura, H. p16(INK4) is inactivated by extensive CpG methylation in human hepatocellular carcinoma. Gastroenterology, 116: 394-400, 1999.
9. Zhang, Y. J., Ahsan, H., Chen, Y., Lunn, R. M., Wang, L. Y., Chen, S. Y., Lee, P. H., Chen, C. J., and Santella, R. M. High frequency of promoter hypermethylation of the RASSF1A and p16 genes and its relationship to aflatoxin B1-DNA adducts level in human hepatocellular carcinoma. Mol. Carcinogenesis, 35: 85-92, 2002.
10. Burbee, D. G., Forgacs, E., Zochbauer-Muller, S., Shivakumar, L., Fong, K., Gao, B., Randle, D., Kondo, M., Virmani, A., Bader, S., Sekido, Y., Latif, F., Milchgrub, S., Toyooka, S., Gazdar, A. F., Lerman, M. I., Zabarovsky, E., White, M., and Minna, J. D. Epigenetic inactivation of RASSF1A in lung and breast cancers and malignant phenotype suppression. Journal of the National Cancer Institute, 93: 691-699, 2001.
11. Dammann, R., Takahashi, T., and Pfeifer, G. P. The CpG island of the novel tumor suppressor gene RASSF1A is intensely methylated in primary small cell lung carcinomas. Oncogene, 20: 3563-3567, 2001.
12. Lee, M. G., Kim, H. Y., Byun, D. S., Lee, S. J., Lee, C. H., Kim, J. I., Chang, S. G., and Chi, S. G. Frequent epigenetic inactivation of RASSF1A in human bladder carcinoma. Cancer Research, 61: 6688-6692, 2001.
13. Lo, K. W., Kwong, J., Hui, A. B., Chan, S. Y., To, K. F., Chan, A. S., Chow, L. S., Teo, P. M., Johnson, P. J., and Huang, D. P. High frequency of promoter hypermethylation of RASSF1A in nasopharyngeal carcinoma. Cancer Research, 61: 3877-3881, 2001.
14. Yamauchi, H., Aminaka, Y., Yoshida, K., Sun, G., Pi, J., and Waalkes, M. P. Evaluation of DNA damage in patients with arsenic poisoning: urinary 8-hydroxydeoxyguanine. Toxicol. Appl. Pharm., 198: 291-296, 2004.
15. Zhang, Y. J., Chen, Y., Ahsan, H., Lunn, R. M., Chen, S. Y., Lee, P. H., Chen, C. J., and Santella, R. M. Silencing of glutathione S-transferase P1 by promoter hypermethylation and its relationship to environmental chemical carcinogens in hepatocellular carcinoma. Cancer Lett., in press: 2004.
16. Pratico, D., Barry, O. P., Lawson, J. A., Adiyaman, M., Hwang, S. W., Khanapure, S. P., Iuliano, L., Rokach, J., and FitzGerald, G. A. IPF2alpha-I: an index of lipid peroxidation in humans. Proc Natl Acad Sci USA, 95: 3449-3454, 1998.
17. Yang, B., Guo, M., Herman, J. G., and Clark, D. P. Aberrant promoter methylation profiles of tumor suppressor genes in hepatocellular carcinoma. American Journal of Pathology, 163: 1101-1107, 2003.
18. Wong, I. H., Lo, Y. M., Yeo, W., Lau, W. Y., and Johnson, P. J. Frequent p15 promoter methylation in tumor and peripheral blood from hepatocellular carcinoma patients. Clin Cancer Res, 6: 3516-3521, 2000.
19. Yoshikawa, H., Matsubara, K., Qian, G. S., Jackson, P. E., Groopman, J. D., Manning, J. E., Harris, C. C., and Herman, J. G. SOCS-1, a negative regulator of the JAK/STAT pathway, is silenced by methylation in human hepatocellular carcinoma and shows growth-suppression activity. Nat Genet, 28: 29-35, 2001.
20. Lee, T. L., Leung, W. K., Chan, M. W., Ng, E. K., Tong, J. H., Lo, K. W., Chung, S. C., Sung, J. J., and To, K. F. Detection of gene promoter hypermethylation in the tumor and serum of patients with gastric carcinoma. Clinical Cancer Research, 8: 1761-1766, 2002.

### Example 2

### Predicting HCC by Detecting Methylation in Serum DNA

Hepatocellular carcinoma (HCC) is one of the most common and rapidly fatal human malignancies. The almost 500,000 new cases and nearly equivalent number of fatalities illustrates the lack of effective therapeutic alternatives for this disease that is largely diagnosed at an advanced stage; most patients die within one year of diagnosis (1). Chronic hepatitis B and C virus infections are well-documented risk factors for the development of HCC. Several environmental factors including aflatoxin B₁ (AFB₁), a dietary mold contaminant, and polycyclic aromatic hydrocarbons, ubiquitous environmental contaminants, are also associated with the development of HCC (2-4). While HCC incidence is highest in East Asia and Sub-Saharan Africa (1), it is also increasing in U.S. (5) Currently available screening tests to detect smaller and more frequently unifocal (early stage) HCC combine alpha-fetoprotein (AFP) analysis and ultrasound. However, although screening for early detection of HCC has become quite common in clinical practice, its effectiveness remains controversial (6).

As with other cancers, the development of HCC is a complex, multistep process. The molecular pathogenesis of HCC appears to involve multiple genetic aberrations in the molecular control of hepatocyte proliferation, differentiation, and death and the maintenance of genomic integrity. This process is influenced by the cumulative activation and inactivation of oncogenes, tumor suppressor genes and other genes. Epigenetic alterations are also involved in cancer development and progression (7-9). Methylation of promoter CpG islands is known to inhibit transcriptional initiation and cause permanent silencing of downstream genes. Hypermethylation of p16, a cyclin-dependent kinase inhibitor gene that regulates the cell cycle, has been detected frequently in human cancers (10). p15, another cyclin-dependent kinase inhibitor gene adjacent to p16 on chromosome 9p21, has been postulated to be a tumor suppressor modulating pRb phosphorylation. It is also aberrantly methylated in several human neoplasm including HCC (11,12). The ras association domain family 1A (RASSF1A) gene is located in chromosome 3p21.3 and, from initial studies in lung and breast cancer, was suggested to be a tumor suppressor gene (13). We and others have consistently reported a high frequency, in the range of 80-90%, of RASSF1A promoter hypermethylation in HCC tissues (14,15).

Detection of methylated DNA has been suggested as a potential biomarker for early detection of cancer (16). Since an ideal biomarker should appear early in the course of disease and should be detectable in biological samples that can be obtained noninvasively, many studies have focused on the detection of genetic and epigenetic abnormalities in exfoliated cells from sputum, bronchoalveolar lavage or cervical smears as well as in the circulating DNA found in serum or plasma. In our recent study, p16 was methylated in 24 of 40 (62%) tissues and 12 of 39 (32%) plasma DNAs from blood collected at the time of diagnosis (17). Other studies of HCC patients have also found that methylated DNA can be frequently detected in blood collected at the time of diagnosis (12, 18, 19). These results suggest that biomarkers in plasma or serum may help in estimating the risk for the development of HCC, however, their sensitivity and specificity for HCC detection, and their clinical utility remain uncertain at the present time. No studies of HCC have assayed blood samples collected years prior to diagnosis for aberrant methylation.

In the present study, we explored the possible diagnostic value of aberrant promoter hypermethylation using a panel of three tumor suppressor genes in serum DNA for early detection of HCC. We took advantage of a sample bank collected for a cancer screening program in Taiwan in which repeat samples prior to diagnosis were available.

### Materials and Methods

Human Subjects and Sample Collection. This study was approved by Columbia University's Institutional Review Board as well as the research ethics committee of the College of Public Health, National Taiwan University, Taipei, Taiwan; written informed consent was obtained from all subjects, and strict quality controls and safeguards were used to protect confidentiality. Fifty subjects with HCC were randomly chosen from cases identified in the Cancer Screening Program study, a community-based cohort recruited in Taiwan; 50 controls were selected by matching by age (within three years) and sex. Blood samples for controls were collected 1991-1192. The cohort characteristics and methods of screening and follow up have been described in more detail previously (20). Briefly, individuals were between 30 to 64 years old and lived in seven townships in Taiwan, three located on Penghu islets with the highest HCC incidence in Taiwan, and the other four from Taiwan island. A total of 12,020 males and 11,923 females were recruited between July 1990 and June 1992. Participants were personally interviewed based on a structured questionnaire and donated a 20 ml blood sample at recruitment. Aliquots of serum were separated from other components in blood and stored at −70° C. Specimens were transported on dry ice to a central laboratory at the National Taiwan University and were kept at −70° C. until shipment to Columbia for analysis.

Blood samples were screened in Taiwan for serological markers including alanine transaminase (ALT), aspartate transaminase (AST), AFP, HBsAg and anti-HCV using commercial kits (HBsAg, anti-HCV and AFP, Abbott Laboratories, North Chicago, Ill., USA) or a serum chemistry autoanalyzer (ALT, AST, Hitachi Model 736; Hitachi Co., Tokyo, Japan). Any participant who had an elevated level of AFP (≧20 ng/ml), was positive for HBsAg or anti-HCV or, had a family history of HCC or liver cirrhosis among first degree relatives was referred for upper abdominal ultrasonographic examination. They were also followed with additional blood collections. Suspected HCC cases were referred to teaching medical centers for confirmatory diagnosis by computerized tomography, digital subtracted angiogram, aspiration cytology and pathological examination. The criteria for HCC diagnosis included: a histo-pathological examination or a positive lesion detected by at least two different imaging techniques. Through 2003, a total of 162 HCC patients provided a baseline and at least one follow-up blood sample. Since the annual follow-up in high risk subjects was voluntary, each case had multiple samples collected prior to diagnosis.

Methylation specific PCR (MSP). DNA was extracted from 200 μl of serum using QIAamp UltraSens Virus Kits (Qiagen, Valencia Calif.) following the viral RNA and DNA purification protocol. Bisulfite modification was conducted using a CpGenome™ DNA Modification Kit (Chemicon International, Temecula Calif.) following the manufacturer's recommendations. PCR was conducted with the CpGWIZ p16 and p15 Amplification Kits (Chemicon International) and AmpliTaq Gold Polymerase (Perkin-Elmer, Norwalk, Conn.) and a total of 40 cycles. The thermal profile consisted of an initial denaturation step of 95° C. for 10 min, followed by repetitions of 95° C. for 45 s, 60° C. for 45 s, and 72° C. for 60 s, with a final extension step of 72° C. for 10 min. For detection of RASSF1A methylation, primers and amplification conditions were as described previously (19). PCR products were analyzed by agarose gel electrophoresis and Vista Green (Amersham Biosciences, Piscataway, N.J.) staining. The methylated DNA control from the CpGnome Amplification Kit and universal methylated DNA (Chemicon International) were used as positive controls and distilled water as a negative control. As a quality control for the bisulfite modification process, all bisulfite-treated DNAs were also amplified with primers specific for the unmethylated p16, p15 and RASSF1A (12, 18, 19).

Statistical analysis. The associations of methylation status with clinical factors were analyzed by Fisher's exact test, using the data from the blood collected closest to diagnosis. Differences in the means of continuous variables (i.e., ALT, AST and AFP) between the methylation statuses of genes were analyzed using Mann-Whitney test. Differences at p<0.05 were considered significant. Conditional logistic regression was used to construct receiver operating characteristic (ROC) curves using clinical risk factors and methylation biomarkers (21).

### Results

Subject characteristics. The demographic data are presented in Table 2. There were a total of 11 females and 39 males, 50% were smokers, 24% had habitual alcohol consumption, 51% were HBsAg positive and 24% were anti-HCV positive.

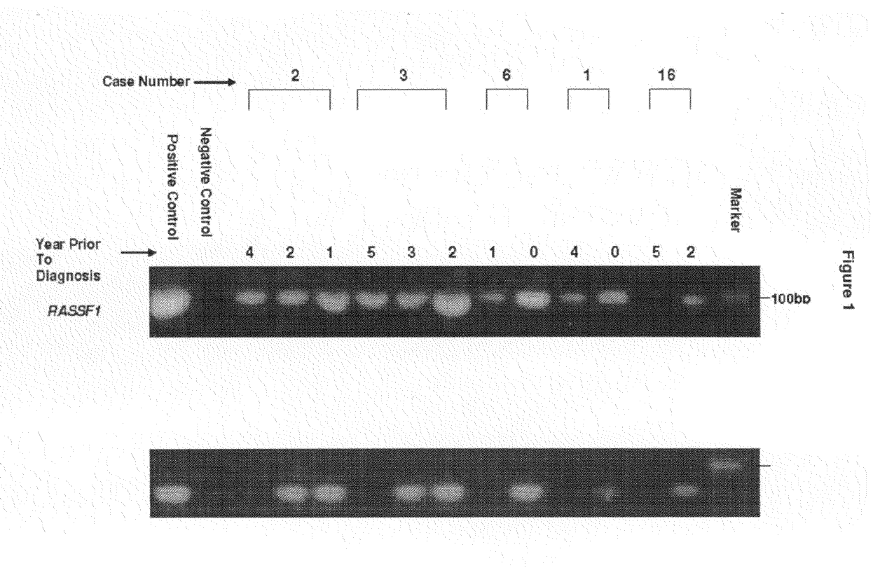

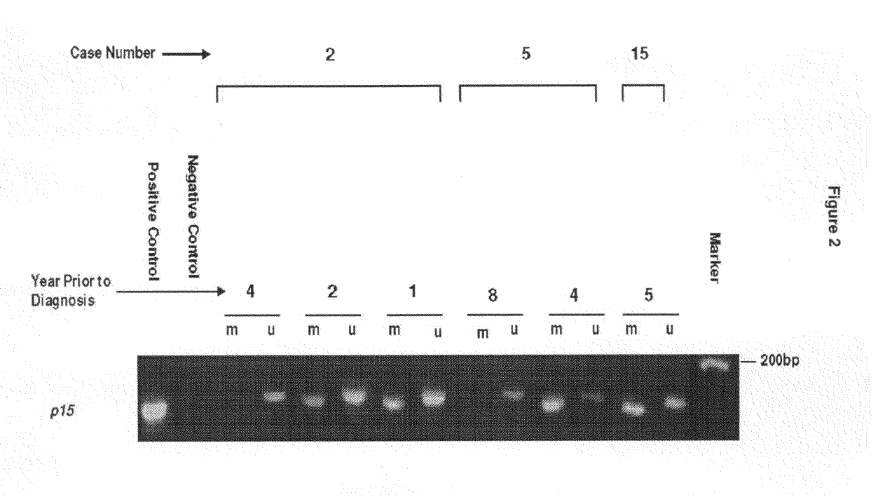

P16, p15 and RASSF1A promoter methylation in serum DNA. The promoter methylation status for p16, p15 and RASSF1A of DNA isolated from serum collected at different time points before diagnosis was assayed by methylation specific PCR (MSP). FIG. 1 shows representative MSP analyses for RASSF1A and p16 in serum DNA from 5 HCC cases with blood collected at different time point prior to diagnosis. In FIG. 2 representative MSP analysis using methylated and unmethylated primers for p15 are shown for three cases. In the 50 serum samples from HCC cases collected closest to diagnosis (0-9 years prior), 22 (44%) were positive for methylation of p16, 12 (22%) for p15 and 35 (70%) for RASSF1A (Table 2). Six of the 50 cases had hypermethylation of all three genes, 13 cases for two genes and 25 cases for one gene; 6 subjects were hypermethylation negative for all three genes.

A total of 14 samples were available that had been collected one to three years earlier than the sample collected closest to diagnosis and of these, hypermethylation was positive for 9 (64%) for p16, 2 (14%) for p15 and 4 (29%) for RASSF1A (Table 3). In the 3 available serum samples that were collected another two years earlier, 2 (67%) were positive for promoter hypermethylation of RASSF1A (Table 3) but none for p16 and p15 (Table 3). The methylation status of p16, p15 or RASSF1A did not differ by gender. The frequency of p16 promoter hypermethylation was significantly higher in HBsAg positive (60.0%) than negative (25.0%) HCC cases (p=0.01) The association of p15 promoter hypermethylation with HBV infection was also statistically significant. (HBsAg positive cases 36.0%, HBsAg negative cases 12.5%) (p=0.04). The association between p16 and p15 promoter hypermethylation was weak and not statistically significant (p=0.05). There was no association of RASSF1A promoter hypermethylation with HBV infection (HBsAg positive case 72.0% and HBsAg negative cases 70.8%) (p=0.24). Methylation status of any one of three genes did not differ based on anti-HCV status, smoking or habitual alcohol consumption. We also looked at the association of methylation and subjects' ALT, AST and AFP status, but no significant relationships were found.

Among the 50 samples from controls, promoter hypermethylation was detected in 3 subjects for RASSF1A and 2 for p16 (Table 4). No subjects were positive for p15 methylation.

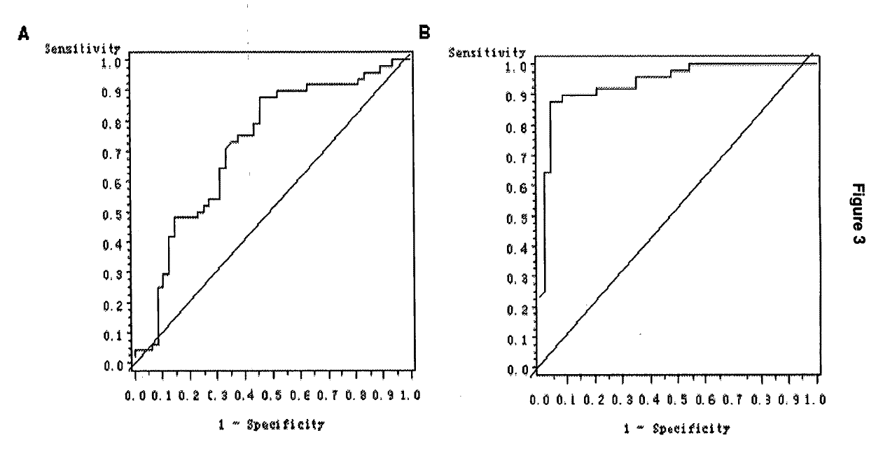

Two ROC curves were constructed by separately including only clinical risk factors (age, HBsAg status, anti-HCV status, smoking, alcohol status) and these factors plus the three hypermethylation biomarkers (p16, p15 and RASSF1 methylation) (FIGS. 3A and B, respectively). The overall predictive accuracy is relatively low (67%) for the model that includes only the clinical risk factors (with a sensitivity of 66% and a specificity of 68%). The overall predictive accuracy is much better (89%) for the model that includes not only clinical factors, but also hypermethylation biomarkers. The sensitivity and specificity were 84% and 94%, respectively, under the probability cut-point of 0.50.

### Discussion

In the present study, we investigated serum DNA methylation for p16, p15 and RASSF1A, three tumor suppressor genes frequently hypermethylated in HCC. Blood samples were collected from 50 HCC cases 0-9 years prior to diagnosis. The frequencies of detection of gene methylation in the available samples collected closest to diagnosis are consistent with previous studies of serum DNA from HCC patients using blood collected at the time of diagnosis: p16, 44.% versus 48% (22), p15, 22% versus 25% (12), and RASSF1A, 70% versus 43% (19). The detection frequencies for p16 and RASSF1A are also similar to our previous findings in HCC tissue DNAs (14). Hypermethylation was detected 1-8 years before clinical diagnosis for p16, 1-5 years for p15; and 1-9 years for RASSF1A. These findings demonstrate that p16, p15 and RASSF1A hypermethylation are early events in the development of HCC.

A specific missense mutation in the p53 tumor suppressor gene at codon 249 has been reported in over 50% of HCC tumors and in paired blood samples from areas of high dietary exposure to AFB₁ (23). Jackson et al. detected this mutation in DNA from plasma collected 1-5 years prior to diagnosis, suggesting that it could be used as a biomarker for aflatoxin exposure and HCC development (24). But it only can be detected in cases from areas with high AFB₁ exposure.

Several studies have indicated that epigenetic changes might ‘addict’ cancer cells to altered signal-transduction pathways during the early stages of tumor development (16,25). Hypermethylation of CpG islands in gene promoters can appear early in the progression of lung and colon cancer or can be characteristic of premalignant lesions at these sites (26). Belinsky et al. reported that the frequency of aberrant methylation of p16 increased during disease progression from bronchial basal cell hyperplasia (17%) to squamous metaplasia (24%) to carcinoma in situ (50%) (27). Aberrant promoter methylation of p16 and MGMT was also detected by others in sputum DNA in 100% of patients with squamous cell carcinoma of the lung up to 3 years before clinical diagnosis (28) and by Belinsky et al. in multiple other genes in sputum from patients with lung cancer several months to 3 years before clinical diagnosis (29). These findings show the promise of gene promoter hypermethylation in sputum as a molecular marker for identifying people at high risk for cancer.

Epigenetic alterations, including methylation of p16 (30-32), p15 (12), RASSF1A (14), MGMT (33), GSTP1 (34,35) and other genes, are prevalent in HCC tissue samples (36). By using MSP, methylation changes of p16, p15 and RASSF1A were also detected in the plasma and serum of HCC patients (13, 19, 20). Wong and colleagues reported a good correlation between p16 hypermethylation in HCC tissues and plasma/serum DNA (72%) and that p16 hypermethylation was not detected in the plasma/serum of patients with either liver cirrhosis or hepatitis (18). However, in another study, 17% of cirrhosis patients had serum DNA with aberrant p16 methylation (22). These differing results may be due to the lack of standardized processing of blood samples and methods of analysis; the relatively small sample size and diversity in the clinical courses of patients may also contribute to the variation. Thus far, no studies on the relationship between methylation status of p15 and RASSF1A in serum DNA and cirrhosis have been reported. Thus, the relationship between methylation status of different tumor suppressor genes and precancerous lesions like cirrhosis needs further study and the significance of epigenetic changes in serum DNA from cirrhosis patients is currently unclear.

p16 and p15 methylation were associated with HBV infection in this study, implying an environment-epigenetic interaction in the development of HCC. A recent study with similar results suggested that hepatitis viruses might induce p16 methylation in liver tissues with chronic inflammation, prior to the appearance of HCC (37), but this correlation is still controversial (14, 18, 38). No correlation between p15 methylation and HBV infection was found in the previous study (12). In the present study, p15 methylation correlated with p16 methylation in serum DNA (p=0.05). Dual p15 and p16 methylation has been found almost exclusively in hematological malignancies such as Burkitt's lymphoma and acute T-cell leukemia (11,39). In terms of clinical relevance, p16 and p15 methylation were significantly associated with the development of a recurrence or metastasis (12). Thus, p16 and p15 methylation may be implicated in tumor progression. While it is recognized that malignant tumors harbor dense methylation in normally unmethylated promoter CpG islands (8), our previous study and those of others demonstrated that hypermethylation of tumor suppressor genes, including p16 and RASSF1A, were absent or very low in normal tissues DNA (14, 40, 41). In a study of breast cancer, a small number of healthy controls (n=10) were tested and RASSF1A was methylated in the plasma DNA from one subject (10%) (42).

In the present study, 50 matched serum DNAs from normal controls were also investigated for methylation status. Promoter hypermethylation of p16 and RASSF1A was detected in 2 and 3 normal controls, respectively. Compared with the 50 cases, these detection levels are very low (2 controls/22 cases for p16 and 3 controls/35 cases for RASSF1A). Three of four positive controls (one subject had hypermethylation in both p16 and RASSF1A) had either HBV or/and HCV infections; one subject had a history of smoking and alcohol drinking. Although it is controversial, some risk factors have been reported to correlate with gene methylation (14,29). Hypermethylation in serum DNA from controls was perhaps due to hepatitis virus infection and chemical carcinogen exposure. Another possibility was that some “normal controls” have cryptogenic hepatic cirrhosis. A small percentage of patients with liver cirrhosis were reported to be positive for p16 methylation in serum DNA (22), but these changes still need further study. In the present study, the 50 cases were randomly selected. Thus, although, the results may not represent the methylation status of all the HCC cases in this cohort, there should be no selection bias.

In conclusion, this is the first study to prospectively examine epigenetic changes in tumor suppressor genes for predicting HCC development in a cohort of high-risk subjects. p16, p15 and RASSF1A promoter hypermethylation were detected in DNA from serum samples collected up to 9 years before clinical diagnosis. Compared to controls, detection of promoter hypermethylation on these three genes was much more frequent in HCC patients prior to diagnosis. These molecular changes may be a valuable biomarker for early detection, risk assessment in high-risk populations and monitoring the clinical course of HCC.

TABLE 2
Demographics, HCV Status, HBsAg Status, and p16,
p15, and RASSF1A Methylation Status of HCC Cases

| ID | Age | Gender | HBsAg | AntiHCV | Smoking | Alcohol | p16^{§} | p15^{§} | RASSF1A^{§} |
|---|---|---|---|---|---|---|---|---|---|
| 1 | 62 | M | + | − | Yes | No | + | + | + |
| 2 | 59 | M | + | − | No | No | + | + | + |
| 3 | 49 | M | + | − | No | Yes | + | + | + |
| 4 | 53 | M | + | − | No | No | + | + | + |
| 5 | 59 | F | + | − | No | No | + | + | + |
| 6 | 57 | M | + | − | Yes | Yes | + | + | + |
| 7 | 63 | M | + | + | Yes | No | + | − | + |
| 8 | 58 | M | + | − | No | No | + | − | + |
| 9 | 63 | F | − | − | No | No | + | − | + |
| 10 | 64 | M | − | − | No | Yes | + | − | + |
| 11 | 62 | M | − | + | No | No | + | − | + |
| 12 | 63 | M | − | + | Yes | No | + | − | + |
| 13 | 59 | M | + | NA* | Yes | No | + | + | − |
| 14 | 53 | M | − | + | Yes | No | − | + | + |
| 15 | 49 | M | + | − | Yes | No | + | + | − |
| 16 | 63 | F | + | + | No | No | + | − | + |
| 17 | 63 | F | − | + | No | No | − | + | + |
| 18 | 57 | F | + | − | No | No | − | + | + |
| 19 | 51 | F | + | − | No | No | + | − | + |
| 20 | 51 | M | − | − | No | Yes | − | − | + |
| 21 | 52 | M | + | − | No | No | − | − | + |
| 22 | 36 | M | + | − | No | No | − | − | + |
| 23 | 56 | M | + | − | Yes | No | − | − | + |
| 24 | 42 | M | − | + | Yes | No | − | − | + |
| 25 | 43 | M | − | + | Yes | Yes | − | − | + |
| 26 | 57 | M | + | − | No | No | + | − | − |
| 27 | 35 | M | − | − | Yes | Yes | − | − | + |
| 28 | 40 | M | + | − | Yes | No | + | − | − |
| 29 | 61 | M | − | − | Yes | No | − | − | + |
| 30 | 60 | M | − | − | Yes | Yes | − | − | + |
| 31 | 61 | M | − | − | Yes | No | − | − | + |
| 32 | 61 | M | − | − | Yes | No | + | − | − |
| 33 | 39 | M | + | − | Yes | No | − | − | + |
| 34 | 56 | M | NA | − | Yes | Yes | + | − | − |
| 35 | 35 | M | + | − | No | No | − | − | + |
| 36 | 63 | M | − | − | Yes | No | − | − | + |
| 37 | 60 | M | − | − | Yes | Yes | − | − | + |
| 38 | 61 | F | + | − | No | No | − | − | + |
| 39 | 63 | F | − | + | No | No | − | + | − |
| 40 | 56 | F | − | + | No | No | + | − | − |
| 41 | 39 | M | − | − | No | No | − | − | + |
| 42 | 64 | M | + | − | No | No | + | − | − |
| 43 | 45 | M | − | + | Yes | No | − | − | + |
| 44 | 49 | M | + | − | No | Yes | − | − | + |
| 45 | 50 | M | + | − | Yes | Yes | − | − | − |
| 46 | 63 | M | + | − | Yes | No | − | − | − |
| 47 | 56 | M | − | − | Yes | Yes | − | − | − |
| 48 | 52 | F | − | − | No | No | − | − | − |
| 49 | 64 | F | − | − | No | No | − | − | − |
| 50 | 47 | M | − | + | Yes | No | − | − | − |

*NA, data not available
^{§}+, methylation positive
−, methylation negative

TABLE 3
p16, p15 and RASSF1A Methylation Status In Serum
DNA From HCC Patients Prior To Diagnosis

| | Year | | | | Year |
|---|---|---|---|---|---|
| | enrolled | Test 1 | Test 2 | Test 3 | diagnosed |
| 1 | 1992 | 1993□⋄ | 1996▪♦ | | 1996 |
| 2 | 1991 | 1993□⋄ | 1995▪♦ | 1996▪♦ | 1997 |
| 3 | 1992 | 1993□⋄ | 1995▪♦ | 1996▪♦ | 1998 |
| 4 | 1992 | 1993▪♦ | | | 1995 |
| 5 | 1991 | 1993▪⋄◯ | 1996▪♦ | | 2000 |
| 6 | 1991 | 1996▪⋄ | 1997▪♦ | | 1997 |
| 7 | 1991 | 1992▪⋄ | | | 1996 |
| 8 | 1992 | 1995▪⋄ | | | 1997 |
| 9 | 1992 | 1993▪⋄◯ | 1995▪⋄ | | 2000 |
| 10 | 1992 | 1993▪⋄ | | | 1999 |
| 11 | 1992 | 1993▪⋄ | | | 2001 |
| 12 | 1992 | 1993▪⋄◯ | 1996▪⋄ | | 1996 |
| 13 | 1991 | 1996▪♦◯ | | | 2000 |
| 14 | 1991 | 1996□♦ | | | 1999 |
| 15 | 1991 | 1992▪♦◯ | | | 1997 |
| 16 | 1992 | 1993□⋄◯ | 1996▪⋄ | | 1998 |
| 17 | 1991 | 1996□♦ | | | 2001 |
| 18 | 1991 | 1996□♦ | | | 1998 |
| 19 | 1991 | 1996▪⋄ | | | 2003 |
| 20 | 1991 | 1993□⋄ | | | 1996 |
| 21 | 1992 | 1996□⋄ | | | 1997 |
| 22 | 1992 | 1995□⋄ | | | 1996 |
| 23 | 1992 | 1996□⋄ | | | 1997 |
| 24 | 1992 | 1996□⋄ | | | 2003 |
| 25 | 1991 | 1996□⋄ | | | 1996 |
| 26 | 1991 | 1995□⋄◯ | 1996▪⋄◯ | | 1996 |
| 27 | 1992 | 1994□⋄ | | | 2000 |
| 28 | 1992 | 1993□⋄◯ | 1995▪⋄◯ | 1996▪⋄◯ | 2000 |
| 29 | 1991 | 1993□⋄ | | | 2000 |
| 30 | 1991 | 1993□⋄ | | | 2000 |
| 31 | 1992 | 1993□⋄ | | | 1997 |
| 32 | 1992 | 1993▪⋄◯ | | | 1997 |
| 33 | 1992 | 1995□⋄ | | | 2000 |
| 34 | 1992 | 1993▪⋄◯ | | | 1999 |
| 35 | 1991 | 1992□⋄ | | | 2001 |
| 36 | 1991 | 1993□⋄ | | | 2000 |
| 37 | 1991 | 1993□⋄ | | | 1995 |
| 38 | 1991 | 1995□⋄ | | | 1996 |
| 39 | 1991 | 1992□⋄◯ | 1996□♦◯ | | 2000 |
| 40 | 1991 | 1996▪⋄◯ | | | 2001 |
| 41 | 1992 | 1995□⋄ | | | 2001 |
| 42 | 1992 | 1995▪⋄◯ | 1996▪⋄◯ | | 2000 |
| 43 | 1991 | 1995□⋄ | | | 2001 |
| 44 | 1991 | 1996□⋄ | | | 2002 |
| 45 | 1992 | 1996□⋄◯ | | | 1997 |
| 46 | 1992 | 1996□⋄◯ | | | 1999 |
| 47 | 1991 | 1993□⋄◯ | | | 1999 |
| 48 | 1992 | 1993□⋄◯ | 1996□⋄◯ | | 2001 |
| 49 | 1992 | 1993□⋄◯ | | | 1998 |
| 50 | 1992 | 1992□⋄◯ | 1996□⋄◯ | | 1999 |

□p16 methylation negative
▪p16 methylation positive
⋄p15 methylation negative
♦p15 methylation positive
◯RASSF1A methylation negative
RASSF1A methylation positive

TABLE 4
Demographics, HBsAg, HCV Status, and Methylation Status of Controls

| ID | Age | Gender | HBsAg | AntiHCV | Smoking | Alcohol | p16^{§} | p15^{§} | RASSF1A^{§} |
|---|---|---|---|---|---|---|---|---|---|
| 1 | 51 | M | + | NA | Yes | No | − | | − |
| 2 | 51 | M | + | − | No | No | − | − | − |
| 3 | 50 | M | − | − | No | No | − | − | − |
| 4 | 59 | M | − | − | Yes | Yes | − | − | − |
| 5 | 60 | M | − | − | Yes | Yes | − | − | − |
| 6 | 59 | M | − | + | No | No | − | − | − |
| 7 | 53 | M | + | − | Yes | Yes | + | − | + |
| 8 | 52 | M | − | − | No | No | − | − | − |
| 9 | 58 | M | + | − | Yes | Yes | − | − | − |
| 10 | 56 | M | − | + | Yes | No | − | − | − |
| 11 | 55 | M | − | − | Yes | Yes | − | − | − |
| 12 | 55 | M | − | − | No | No | − | − | − |
| 13 | 53 | M | − | − | Yes | Yes | + | − | − |
| 14 | 51 | M | − | − | No | No | − | − | − |
| 15 | 65 | F | − | − | No | No | − | − | − |
| 16 | 62 | F | − | − | No | No | − | − | − |
| 17 | 64 | F | − | − | No | No | − | − | − |
| 18 | 40 | M | − | − | Yes | Yes | − | − | − |
| 19 | 36 | M | − | − | No | No | − | − | − |
| 20 | 36 | M | − | − | No | No | − | − | − |
| 21 | 37 | M | − | − | Yes | No | − | − | − |
| 22 | 37 | M | − | − | Yes | No | − | − | − |
| 23 | 50 | M | − | − | No | No | − | − | − |
| 24 | 57 | M | − | − | Yes | No | − | − | − |
| 25 | 58 | M | − | − | Yes | No | − | − | − |
| 26 | 57 | M | − | − | Yes | No | − | − | − |
| 27 | 43 | M | − | − | No | Yes | − | − | − |
| 28 | 40 | M | − | − | Yes | No | − | − | − |
| 29 | 42 | M | − | − | No | No | − | − | − |
| 30 | 48 | M | + | + | Yes | No | − | − | − |
| 31 | 60 | M | − | − | No | No | − | − | − |
| 32 | 63 | M | − | − | Yes | No | − | − | − |
| 33 | 64 | M | − | − | Yes | No | − | − | − |
| 34 | 58 | M | + | − | Yes | No | − | − | − |
| 35 | 55 | M | − | − | Yes | Yes | − | − | − |
| 36 | 55 | F | − | NA* | No | No | − | − | + |
| 37 | 45 | M | + | + | Yes | Yes | − | − | − |
| 38 | 55 | M | − | − | Yes | Yes | − | − | − |
| 39 | 60 | M | − | + | No | No | − | − | − |
| 40 | 45 | M | + | − | No | No | − | − | − |
| 41 | 56 | M | − | + | Yes | No | − | − | − |
| 42 | 45 | M | + | − | No | No | − | − | − |
| 43 | 48 | M | + | − | No | No | − | − | − |
| 44 | 63 | F | − | − | No | No | − | − | − |
| 45 | 62 | F | − | − | No | No | − | − | − |
| 46 | 64 | F | − | + | No | No | − | − | − |
| 47 | 64 | F | − | − | No | No | − | − | − |
| 48 | 62 | F | − | − | No | No | − | − | − |
| 49 | 60 | F | − | + | No | No | − | − | + |
| 50 | 62 | F | + | − | No | No | − | − | − |

*NA, data not available
^{§}+, methylation positive;
−, methylation negative

### REFERENCES

1. Schafer D F, Sorrell M F. Hepatocellular carcinoma. Lancet 1999; 353:1253-7.
2. Chen C J, Yu M W, Liaw Y F. Epidemiologic characteristics and risk factors of hepatocellular carcinoma. J Gastroenterol Hepatol 1997; 12:S294-308.
3. Santella R M, Zhang Y J, Hsieh L L, Young T L, Lu X Q, Lee B M, Yang G Y, and Perera F P. Immunological methods for monitoring human exposure to benzo[α]pyrene and aflatoxin B1: measurement of carcinogen adducts. In: M. Vanderlaan (ed.), Immunoassays for Monitoring human exposure to Toxic Chemicals, pp. 229-245. Washington, D.C.: ACS Publications, 1991.
4. Chen S Y, Wang L Y, Lunn R, et al. Polycyclic aromatic hydrocarbon-DNA adducts in liver tissues of hepatocellular carcinoma patients and controls. International J Cancer 2002; 99:14-21.
5. El-Serag H B, Mason A C. Rising incidence of hepatocellular carcinoma in the United States. New England Journal of Medicine 1999; 340:745-50.
6. De Masi S, Tosti M E, Mele A. Screening for hepatocellular carcinoma. Dig Liver Dis 2005; 37:260-8.
7. Jones P A, Laird P W. Cancer epigenetics comes of age. Nature Genetics 1999; 21:163-7.
8. Baylin S B, Herman J G. DNA hypermethylation in tumorigenesis: epigenetics joins genetics. Trends in Genetics 2000; 16:168-74.
9. Feinberg A P. Cancer epigenetics takes center stage. Proc Natl Acad Sci USA 2001; 98:392-4.
10. Esteller M, Corn P G, Baylin S B, et al. A gene hypermethylation profile of human cancer. Cancer Res 2001; 61:3225-9.
11. Herman J G, Civin C I, Issa J P, et al. Distinct patterns of inactivation of p15INK4B and p16INK4A characterize the major types of hematological malignancies. Cancer Res 1997; 57:837-41.
12. Wong I H, Lo Y M, Yeo W, et al. Frequent p15 promoter methylation in tumor and peripheral blood from hepatocellular carcinoma patients. Clin Cancer Res 2000; 6:3516-21.
13. Dammann R, Li C, Yoon J H, et al. Epigenetic inactivation of a RAS association domain family protein from the lung tumour suppressor locus 3p21.3. Nature Genetics 2000; 25:315-9.
14. Zhang Y J, Ahsan H, Chen Y, et al. High frequency of promoter hypermethylation of the RASSF1A and p16 genes and its relationship to aflatoxin B1-DNA adducts level in human hepatocellular carcinoma. Mol Carcinogenesis 2002; 35:85-92.
15. Zhong S, Yeo W, Tang M W, et al. Intensive hypermethylation of the CpG island of Ras association domain family 1A in hepatitis B virus-associated hepatocellular carcinomas. Clin Cancer Res 2003; 9:3376-82.
16. Baylin S B, Ohm J E. Epigenetic gene silencing in cancer—a mechanism for early oncogenic pathway addiction? Nat Rev Cancer 2006; 6:107-16.
17. Zhang Y J, Rossner P., Chen Y, et al. Aflatoxin B1 and polycyclic aromatic hydrocarbon adducts, p53 mutations and p16 methylation in liver tissue and plasma of hepatocellular carcinoma patients. International Journal of Cancer 2006; 119:985-91.
18. Wong I H, Lo Y M, Zhang J, et al. Detection of aberrant p16 methylation in the plasma and serum of liver cancer patients. Cancer Res 1999; 59:71-3.
19. Yeo W, Wong N, Wong W L, et al. High frequency of promoter hypermethylation of RASSF1A in tumor and plasma of patients with hepatocellular carcinoma. Liver Int 2005; 25:266-72.
20. Wang L Y, Hatch M, Chen C J, et al. Aflatoxin exposure and the risk of hepatocellular carcinoma in Taiwan. Int J Cancer 1996; 67:620-5.
21. Zweig M H, Campbell G. Receiver-operating characteristic (ROC) plots: a fundamental evaluation tool in clinical medicine. Clin Chem 1993; 39:561-77.
22. Chu H J, Heo J, Seo S B, et al. Detection of aberrant p16INK4A methylation in sera of patients with liver cirrhosis and hepatocellular carcinoma. J Korean Med Sci 2004; 19:83-6.
23. Jackson P E, Qian G S, Friesen M D, et al. Specific p53 mutations detected in plasma and tumors of hepatocellular carcinoma patients by electrospray ionization mass spectrometry. Cancer Res 2001; 61:33-5.
24. Jackson P E, Kuang S Y, Wang J B, et al. Prospective detection of codon 249 mutations in plasma of hepatocellular carcinoma patients. Carcinogenesis 2003; 24:1657-63.
25. Toyota M, Issa J P. Epigenetic changes in solid and hematopoietic tumors. Semin Oncol 2005; 32:521-30.
26. Herman J G, Baylin S B. Gene silencing in cancer in association with promoter hypermethylation. N Engl J Med 2003; 349:2042-54.
27. Belinsky S A, Nikula K J, Palmisano W A, et al. Aberrant methylation of p16(INK4a) is an early event in lung cancer and a potential biomarker for early diagnosis. Proc Natl Acad Sci USA 1998; 95:11891-6.
28. Palmisano W A, Divine K K, Saccomanno G, et al. Predicting lung cancer by detecting aberrant promoter methylation in sputum. Cancer Res 2000; in press.
29. Belinsky S A, Liechty K C, Gentry F D, et al. Promoter hypermethylation of multiple genes in sputum precedes lung cancer incidence in a high-risk cohort. Cancer Res 2006; 66:3338-44.
30. Hui A M, Sakamoto M, Kanai Y, et al. Inactivation of p16^{INK4} in hepatocellular carcinoma. Hepatol 1996; 24:575-9.
31. Liew C T, Li H M, Lo K W, et al. High frequency of p16INK4A gene alterations in hepatocellular carcinoma. Oncogene 1999; 18:789-95.
32. Matsuda Y, Ichida T, Matsuzawa J, et al. p16(INK4) is inactivated by extensive CpG methylation in human hepatocellular carcinoma. Gastroenterology 1999; 116:394-400.
33. Zhang Y J, Chen Y, Ahsan H, et al. Inactivation of the DNA repair gene O⁶-methylguanine-DNA methyltransferase by promoter hypermethylation and its relationship to aflatoxin B₁-DNA adducts and p53 mutations in hepatocellular carcinoma. Int J Cancer 2003; 103:440-4.
34. Zhong S, Tang M W, Yeo W, et al. Silencing of GSTP1 gene by CpG island DNA hypermethylation in HBV-associated hepatocellular carcinomas. Clin Cancer Res 2002; 8:1087-92.
35. Zhang Y J, Chen Y, Ahsan H, et al. Silencing of glutathione S-transferase P1 by promoter hypermethylation and its relationship to environmental chemical carcinogens in hepatocellular carcinoma. Cancer Lett 2005; 221:135-43.
36. Shen L, Ahuja N, Shen Y, et al. DNA methylation and environmental exposures in human hepatocellular carcinoma. Journal of the National Cancer Institute 2002; 94:755-61.
37. Narimatsu T, Tamori A, Koh N, et al. p16 promoter hypermethylation in human hepatocellular carcinoma with or without hepatitis virus infection. Intervirology 2004; 47:26-31.
38. Shim Y H, Yoon G S, Choi H J, et al. p16 Hypermethylation in the early stage of hepatitis B virus-associated hepatocarcinogenesis. Cancer Lett 2003; 190:213-9.
39. Batova A, Diccianni M B, Yu J C, et al. Frequent and selective methylation of p15 and deletion of both p15 and p16 in T-cell acute lymphoblastic leukemia. Cancer Res 1997; 57:832-6.
40. Krassenstein R, Sauter E, Dulaimi E, et al. Detection of breast cancer in nipple aspirate fluid by CpG island hypermethylation. Clin Cancer Res 2004; 10:28-32.
41. Hibi K, Koike M, Nakayama H, et al. A cancer-prone case with a background of methylation of p16 tumor suppressor gene. Clin Cancer Res 2003; 9:1053-6.
42. Muller H M, Widschwendter A, Fiegl H, et al. DNA methylation in serum of breast cancer patients: an independent prognostic marker. Cancer Research 2003; 63:7641-5.

### Example 3

### Plasma DNA Methylation as an Early Biomarker for Prostate Cancer

Epigenetic alterations are now well established in cancer development and progression.¹⁻³ Methylation of promoter CpG islands is known to inhibit transcriptional initiation and cause permanent silencing of downstream genes. In prostate cancer, hypermethylation of the promoter regions of a number of genes including GSTP1, RASSF1A and RARβ2 has been detected although different methods of detection found different frequencies (reviewed in ⁴). Most recently, the presence of aberrant methylation in urinary cells obtained after prostate massage was found to be associated with prostate cancer. A panel of 4 genes (GSTP1, APC, RASSF1A and RARβ2) could stratify patients into low and high risk of having prostate cancer with a sensitivity of 86% and a diagnostic accuracy of 89%.⁵

Methylation has also been reported in benign prostatic hyperplasia (BPH), sometimes with similar frequency to that observed in prostate cancer.⁶ However, assays that can quantitate the level of methylation have been suggested to be able to discriminate between benign tissue and carcinoma. For example, APC was reported to be methylated in 100% of tumors and 87% of BPH (although another publication reported 65 and 7%, respectively⁷) but the median levels of methylation detected were significantly different (86 and 0.7, respectively).⁸

Detection of hypermethylated DNA has been suggested as a potential biomarker for early detection of cancer.⁹ Since an ideal biomarker should appear early in the course of disease and should be detectable in biological samples that can be obtained noninvasively, many studies have focused on the detection of genetic and epigenetic abnormalities in exfoliated cells from sputum, bronchoalveolar lavage or cervical smears as well as in the circulating DNA found in serum or plasma. Few papers have reported on the presence of methylated DNA in the serum or plasma of prostate cancer patients. In one study, the presence of GSTP1 promoter hypermethylation was found in plasma DNA of 12% of men with clinically localized disease and 28% of men with metastatic cancer¹⁰ while in another 75% of newly diagnosed men were positive.¹¹

In addition to gene specific hypermethylation, global hypomethylation is also frequent in prostate and other cancers. Hypomethylation results in transcriptional activation of repetitive sequences leading to disruption of gene expression. It also facilitates genomic instability.⁶ Significant levels of hypomethylation, including that of LINE-1 retrotransponsons, have been observed in prostate cancer.^{6,16-19} There are no studies of global hypomethylation in plasma DNA in prostate cancer.

We propose to take advantage of the biospecimens and end of study biopsy information to determine if plasma DNA can be used for the early diagnosis of prostate cancer. We will determine the frequency of methylation in a panel of genes previously found to be methylated in prostate cancer and in controls. They will include GSTP1, RASSF1A, RARβ2, APC, p16, TNFRSF10C, BCL2, MDR1, ASC, MGMT, DAPK, MT1G, CDH1, PTGS2 and TIG1.^{5,7} Receiver operator curves will be constructed to determine the sensitivity, specificity and predictive accuracy of the biomarkers. We will also investigate levels of global hypomethylation in the samples.

Using plasma banked during the white blood cell collection, we propose to:

Aim 1 Determine the frequency of gene specific promoter methylation for a panel of genes in the 567 cases diagnosed >one month after sample collection and in an equal number of PCPT participants with negative end of study biopsy. These controls will be matched to cases in terms of age and race/ethnicity

Aim 2 Determine global levels of methylation in these same samples.

As shown in Table 5, a total of 567 subjects provided a blood sample at least one month prior to diagnosis.

TABLE 5
Time of White Blood Cell Collection
in Relation to Date of Diagnosis

| | N | Percent | | |
|---|---|---|---|---|
| After diagnosis | 237 | 24.69 | | |
| Day of diagnosis | 100 | 10.42 | | |
| 1-30 days prior | 56 | 5.83 | | |
| 31-181 days prior | 111 | 11.56 | | |
| 6 mo-1 year prior | 194 | 20.21 | | |
| 1-2 years prior | 196 | 20.42 | {close oversize bracket} | 567 |
| 2-3 years prior | 62 | 6.46 | | |
| >3 years prior | 4 | 0.42 | | |
| Total | 960 | 100.00 | | |

### Requirements of Biological Specimens:

A total of 300 μl of plasma (out of the ˜10 ml originally collected) from each subject will be used to isolate DNA.

### Requirements of Clinical Data:

We will need basic epidemiologic data from all subjects including age at blood donation, race/ethnicity, smoking status, and alcohol consumption. For cases, information on tumor size, stage, grade, PSA levels closest to white blood cell collection and Gleason score.

### Number of Samples Requested:

A total of 1134 samples (567 cases and 567 controls) will be requested.

### REFERENCES

1. Jones P A, Laird P W. Cancer epigenetics comes of age. Nature Genetics. 1999; 21:163-167.
2. Baylin S B, Herman J G. DNA hypermethylation in tumorigenesis: epigenetics joins genetics. Trends in Genetics. 2000; 16:168-174.
3. Feinberg A P. Cancer epigenetics takes center stage. Proc Natl Acad Sci USA. 2001; 98:392-394.
4. Perry A S, Foley R, Woodson K et al. The emerging roles of DNA methylation in the clinical management of prostate cancer. [Review] [174 refs]. Endocrine-Related Cancer. 2006; 13:357-377.
5. Roupret M, Hupertan V, Yates D R et al. Molecular detection of localized prostate cancer using quantitative methylation-specific PCR on urinary cells obtained following prostate massage. Clinical Cancer Research. 2007; 13:1720-1725.
6. Perry A S, Foley R, Woodson K et al. The emerging roles of DNA methylation in the clinical management of prostate cancer. Endocrine-Related Cancer. 2006; 13:357-377.
7. Cho N Y, Kim B H, Choi M et al. Hypermethylation of CpG island loci and hypomethylation of LINE-1 and Alu repeats in prostate adenocarcinoma and their relationship to clinicopathological features. Journal of Pathology. 2007; 211:269-277.
8. Jeronimo C, Henrique R, Hogue M O et al. A quantitative promoter methylation profile of prostate cancer. Clinical Cancer Research. 2004; 10:8472-8478.
9. Baylin S B, Ohm J E. Epigenetic gene silencing in cancer—a mechanism for early oncogenic pathway addiction? Nat Rev Cancer. 2006; 6:107-116.
10. Bastian P J, Palapattu G S, Lin X et al. Preoperative serum DNA GSTP1 CpG island hypermethylation and the risk of early prostate-specific antigen recurrence following radical prostatectomy. Clinical Cancer Research. 2005; 11:4037-4043.
11. Papadopoulou E, Davilas E, Sotiriou V et al. Cell-free DNA and RNA in plasma as a new molecular marker for prostate and breast cancer. Annals of the New York Academy of Sciences. 2006; 1075:235-243.
12. Zhang Y J, Chen Y, Ahsan H et al. Inactivation of the DNA repair gene 06-methylguanine-DNA methyltransferase by promoter hypermethylation and its relationship to aflatoxin B1-DNA adducts and p53 mutations in hepatocellular carcinoma. Int J Cancer. 2003; 103:440-444.
13. Zhang Y J, Chen Y, Ahsan H et al. Silencing of glutathione S-transferase P1 by promoter hypermethylation and its relationship to environmental chemical carcinogens in hepatocellular carcinoma. Cancer Lett. 2005; 221:135-143.
14. Zhang Y J, Ahsan H, Chen Y et al. High frequency of promoter hypermethylation of the RASSF1A and p16 genes and its relationship to aflatoxin B1-DNA adducts level in human hepatocellular carcinoma. Mol Carcinogenesis. 2002; 35:85-92.
15. Zhang Y J, Rossner P., Chen Y et al. Aflatoxin B1 and polycyclic aromatic hydrocarbon adducts, p53 mutations and p16 methylation in liver tissue and plasma of hepatocellular carcinoma patients. International Journal of Cancer. 2006; 119:985-991.
16. Bedford M T, van Helden P D, Bedford M T et al. Hypomethylation of DNA in pathological conditions of the human prostate. Cancer Research. 1987; 47:5274-5276.
17. Tokizane T, Shiina H, Igawa M et al. Cytochrome P450 1B1 is overexpressed and regulated by hypomethylation in prostate cancer. Clinical Cancer Research. 2005; 11:5793-5801.
18. Brothman A R, Swanson G, Maxwell T M et al. Global hypomethylation is common in prostate cancer cells: a quantitative predictor for clinical outcome? Cancer Genetics & Cytogenetics. 2005; 156:31-36.
19. Schulz W A, Elo J P, Florl A R et al. Genomewide DNA hypomethylation is associated with alterations on chromosome 8 in prostate carcinoma. Genes, Chromosomes & Cancer. 2002; 35:58-65.

### Example 4

### Plasma DNA Methylation as an Early Biomarker for Breast Cancer

Aberrant gene expression is the hallmark of cancer cells. In addition to classical genetic mechanisms such as deletions and mutations, growth regulatory genes can be inactivated epigenetically via methylation of cytosine-residues in the promoter region of these genes. Hypermethylation of CpG islands in promoter regions is now recognized as an important and early event in carcinogenesis. Detection of methylated DNA in serum or plasma has been suggested to be a marker for early cancer development. In this study, we examined whether tumor DNA can be detected in plasma of blood collected prior to diagnosis of breast cancer in women and their healthy siblings from high risk families. We measured the methylation status of two growth regulatory genes, p16 and RASSF1A in their plasma DNAs. A total of 72 plasma DNAs from 62 women who gave blood prior to diagnosis and 10 of their healthy siblings were isolated using Qiagen kits. After chemical modification of plasma DNA with EZ-modification Kit, we analyzed the methylation pattern in p16 and RASSF1A genes using Methylation Specific PCR (MSP). We found methylation in 90% of subjects for p16 and 31% for RASSF1A among all cases and siblings. All control siblings had methylation in p16 and 4 of 9 in RASSF1A. The high frequency of methylation in controls may be because subjects come from high cancer risk families. Therefore, we are currently determining methylation levels for both genes in age-matched healthy controls who are not from high risk families. The time interval between blood collection and diagnosis ranged from 2 months and 5 years. These results suggest that detection of aberrant promoter hypermethylation in serum/plasma DNA is potentially a powerful approach to screening for early detection of breast cancer cases in high risk populations.

What is claimed is:

1. A method of predicting the occurrence of hepatocellular carcinoma in a subject, comprising the steps of:
(a) preparing DNA samples from blood samples of the subject; and
(b) determining methylation status of a group of genes comprising RASSF1A, p16 and p15, wherein hypermethylation of these genes as compared to normal control samples indicates the subject is likely to develop hepatocellular carcinoma in the future.

2. The method of claim 1, wherein the blood samples are serum or plasma samples.

3. The method of claim 1, wherein the method is preformed at least one year before the occurrence of hepatocellular carcinoma in the subject.

4. The method of claim 1, wherein the subject is in a high risk group for developing hepatocellular carcinoma.

5. The method of claim 1, wherein the hypermethylation occurs at the promoter regions of the genes.

6. A method of predicting the occurrence of breast cancer in a subject, comprising the steps of:
(a) preparing DNA samples from blood samples of the subject; and
(b) determining methylation status of a group of genes comprising RASSF1A and p16, wherein hypermethylation of these genes as compared to normal control samples indicates the subject is likely to develop breast cancer in the future.

7. The method of claim 6, wherein the blood samples are serum or plasma samples.

8. The method of claim 6, wherein the method is preformed at least one year before the occurrence of breast cancer in the subject.

9. The method of claim 6, wherein the hypermethylation occurs at the promoter regions of the genes.

10. A method of predicting the occurrence of prostate cancer in a subject, comprising the steps of:
(a) preparing DNA samples from blood samples of the subject; and
(b) determining methylation status of one or more genes from a group comprising GSTP1, RASSF1A, RARβ2, APC, p16, TNFRSF10C, BCL2, MDR1, ASC, MGMT, DAPK, MT1G, CDH1, PTGS2 and TIG1, wherein hypermethylation of these genes as compared to normal control samples indicates the subject is likely to develop prostate cancer in the future.

11. The method of claim 10, wherein the blood samples are serum or plasma samples.

12. The method of claim 10, wherein the method is preformed at least one year before the occurrence of prostate cancer in the subject.

13. The method of claim 10, wherein the hypermethylation occurs at the promoter regions of the genes.
